(19) United States
(12) Patent Application Publication
(10) Pub. No.: US 2004/0009578 A1
(43) Pub. Date: Jan. 15, 2004

(54) Process for the preparation of L-amino acids using coryneform bacteria which contain an attenuated mez gene

(75) Inventors: Brigitte Bathe, Salzkotten (DE); Mike Farwick, Essen (DE); Achim Marx, Banska Bystrica (SK); Walter Pfefferle, Halle (DE)

Correspondence Address: Michael A. Sanzo, Fitch, Even, Tabin & Flannery, Suite 401L, 1801 K Street, N.W., Washington, DC, 20006-1201, US

(73) Assignee: Degussa AG

(21) Appl. No.: 10/446,154

(22) Filed: May 28, 2003

(30) Foreign Application Priority Data
May 31, 2002 (DE) 102 24 088.4

(51) Int. Cl. C12N 1/20

(52) U.S. Cl. 435/252.3

(57) ABSTRACT

The present invention relates to a process for the preparation of L-amino acids by the fermentation of coryneform bacteria in which the gene which codes for the malate enzyme is attenuated. Optionally, genes of the biosynthesis pathway of the desired L-amino acid may be enhanced in the bacteria or metabolic pathways which reduce the formation of the desired L-amino acid may be diminished.

### CROSS REFERENCE TO RELATED APPLICATIONS

The present application claims priority to German application 102 24 088.4, filed on May 31, 2002, the contents of which is hereby incorporated by reference.

### FIELD OF THE INVENTION

The invention relates to a process for the preparation of L-amino acids using coryneform bacteria in which the mez gene, which codes for the malate enzyme (malic enzyme, EC:1.1.1.40), is attenuated.

### BACKGROUND OF THE INVENTION

L-amino acids, especially L-lysine and L-methionine, are used in animal nutrition, in human medicine and in the pharmaceuticals industry. One effective manner of producing amino acids for these purposes is by the fermentation of strains of coryneform bacteria and, in particular, Corynebacterium glutamicum. Because of its great importance, improvements are constantly being made in this process. Such improvements may relate to fermentation procedures (e.g., the stirring of preparations or supply of oxygen) or to the composition of the nutrient media (e.g., the sugar concentration present during fermentation). Alternatively, improvements may relate to the methods by which product is purified or to the intrinsic synthetic properties of the microorganism itself.

Methods of mutagenesis and selection have been used to increase the amount of amino acid produced by microorganisms. Strains which are resistant to antimetabolites, e.g., the lysine analogue S-(2-aminoethyl)-L-cysteine or that are auxotrophic for metabolites of regulatory importance and produce L-amino acids may be obtained in this manner. In addition, recombinant DNA techniques have been used to improve the production characteristics of Corynebacterium glutamicum strains.

### OBJECT OF THE INVENTION

The object of the present invention is to provide improved procedures for the fermentative preparation of L-amino acids, particularly L-lysine and L-methionine, by coryneform bacteria.

### SUMMARY OF THE INVENTION

The present invention is based upon the development of an improved process for the preparation of L-amino acids by fermenting coryneform bacteria. In this process, bacteria are used that have been modified so that their nucleotide sequence coding for the malate enzyme (malic enzyme; mez gene) is attenuated and, in particular, eliminated or expressed at a low level. The malate enzyme catalyses oxidative decarboxylation of malate to pyruvate, with a molecule of carbon dioxide being split off. The invention also encompasses the modified bacteria used in the processes.

In its first aspect, the invention is directed to a process for producing a desired amino acid by fermenting coryneform bacteria and thereby creating a fermentation broth. The bacteria used have been modified so that their nucleotide sequence coding for the malate enzyme (mez) is attenuated relative to the unmodified or wild-type coryneform bacteria. In the next step, the desired L-amino is concentrated in the fermentation broth or in the fermented bacterial cells and is then purified to make an isolated composition which may optionally include, in addition to the desired amino acid, biomass or constituents from the fermentation broth.

As used herein, the term “L-amino acids” or “amino acids” means one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. A “desired amino acid” is one which the user of the disclosed methods wishes to produce, with L-lysine and L-methionine being particularly preferred. When the terms “L-lysine” or “lysine” are mentioned herein, they will be understood to refer not only to the bases, but also to salts of the amino acids, such as, e.g., lysine monohydrochloride or lysine sulfate. Similarly, when “L-methionine” or “methionine” are mentioned, this includes salts such as, e.g., methionine hydrochloride and methionine sulfate.

In alternative embodiments, the bacteria used in fermentations may contain modifications in addition to those affecting the gene for the malate enzyme as described above. For example, the bacteria may be modified so that at least one gene product in the biosynthetic pathway of the desired L-amino acid is enhanced, i.e., increased, relative to the activity of the corresponding gene product in the wild type or unmodified, coryneform bacteria, e.g., by increasing expression of the corresponding gene. Alternatively, a gene product in a metabolic pathway that reduces the formation of the desired amino acid may be reduced in activity in the modified coryneform bacteria. This may be accomplished either by reducing the protein's biological activity or by reducing gene expression of the protein.

In one set of preferred embodiments, the desired L-amino acid is either L-lysine or L-methionine and, relative to wild type or unmodified coryneform bacteria, the modified coryneform bacteria have an increased enzymatic activity or concentration of one or more of the following proteins: feed-back resistant aspartate kinase, coded for by the lysC gene; dihydrodipicolinate synthase, coded for by the dapA gene; glyceraldehyde 3-phosphate dehydrogenase, coded for by the gap gene; pyruvate carboxylase, coded for by the pyc gene; malate:quinone oxidoreductase, coded for by the mqo gene; glucose 6-phosphate dehydrogenase, coded for by the zwf gene; the lysine export protein, coded for by the lyse; the Zwa1 protein, coded for by the zwa1 gene; triose phosphate isomerase, coded for by the tpi gene; and 3-phosphoglycerate kinase, coded for by the pgk gene. In each case, protein activity can be increased by overexpressing the gene encoding the protein.

In an alternative set of preferred embodiments, the desired L-amino acid is either L-lysine or L-methionine and, relative to wild type coryneform bacteria, the modified coryneform bacteria exhibit decreased enzymatic activity or concentration of one or more of the following proteins: phosphoenolpyruvate carboxykinase, coded for by the pck gene; glucose 6-phosphate isomerase, coded for by the pgi gene; pyruvate oxidase, coded for by the poxB gene; the Zwa2 protein coded for by the zwa2 gene; and catabolite control protein A, coded for by the ccpA1 gene. One way to reduce activity, is by reducing the expression of genes encoding the proteins.

In all cases, the most preferred modified coryneform bacteria and wild type coryneform bacteria are of the species Corynebacterium glutamicum. Corynebacterium glutamicum modified using the plasmid pK18mobsacBdeltamez are of particular use in the invention.

In a different but related aspect, the invention is directed to a modified coryneform bacterium in which the polynucleotide coding for the malate enzyme (mez) is attenuated relative to unmodified or wild-type coryneform bacteria, preferably by reducing mez gene expression. Relative to its wild type counterparts, the modified bacterium may also exhibit increased enzymatic activity or concentration of a protein from the group consisting of: feed-back resistant aspartate kinase, coded for by the lysC gene; dihydrodipicolinate synthase, coded for by the dapA gene; glyceraldehyde 3-phosphate dehydrogenase, coded for by the gap gene; pyruvate carboxylase, coded for by the pyc gene; malate:quinone oxidoreductase, coded for by the mqo gene; glucose 6-phosphate dehydrogenase, coded for by the zwf gene; the lysine export protein, coded for by the lysE; the Zwa1 protein, coded for by the zwa1 gene; triose phosphate isomerase, coded for by the tpi gene; and 3-phosphoglycerate kinase, coded for by the pgk gene. In general, this increased activity or concentration can be achieved by overexpressing the relevant gene.

As an alternative to, or in conjunction with, increasing the activity of the proteins described above, the modified bacterium may exhibit decreased enzymatic activity or concentration of a protein selected from: phosphoenolpyruvate carboxykinase, coded for by the pck gene; glucose 6-phosphate isomerase, coded for by the pgi gene; pyruvate oxidase, coded for by the poxB gene; the Zwa2 protein coded for by the zwa2 gene; and catabolite control protein A, coded for by the ccpA1 gene. These bacteria may be characterized by decreased expression of the genes for one or more of these proteins.

The most preferred bacterium is of the species Corynebacterium glutamicum, with bacteria made using the plasmid pK18mobsacBdeltamez being of particular use.

### BRIEF DESCRIPTION OF THE DRAWINGS

FIG. 1: Map of the plasmid pK18mobsacBdeltamez: The base pair numbers shown in the FIGURE are approximate values obtained in the context of reproducibility of measurements. The abbreviations and designations used have the following meaning:

| | |
|---|---|
| oriV: | ColE1-similar origin from pMB1; |
| sacB: | the sacB gene which codes for the protein levan sucrase; |
| RP4mob: | RP4 mobilization site; |
| Km: | resistance gene for kanamycin; |
| LacZ′: | 5′-terminus of the lacZα gene fragment; |
| LacZ: | 3′-terminus of the lacZα gene fragment; |
| deltamez: | deleted allele of the mez gene from C. glutamicum; |
| BamHI: | cleavage site of the restriction enzyme BamHI; |
| EcoRI: | cleavage site of the restriction enzyme EcoRI. |

### DETAILED DESCRIPTION OF THE INVENTION

The term “attenuation” or “attenuate” as used herein describes a reduction or elimination of the intracellular activity or concentration of one or more enzymes or proteins in a microorganism. This may be accomplished, for example, by putting the gene encoding the protein under the control of a weak promoter, by replacing the gene with a sequence that codes for a corresponding enzyme with a low activity, by inactivating the gene, by directly inactivating the protein, or, optionally, by combining these measures. Using such measures, the activity or concentration of the corresponding protein may be reduced to 0 to 75%, 0 to 50%, 0 to 25%, 0 to 10% or 0 to 5% of the activity or concentration of the wild-type protein or of the activity or concentration of the protein in the starting, i.e., unmodified, microorganism.

The microorganisms provided by the present invention can prepare amino acids from glucose, sucrose, lactose, fructose, maltose, molasses, starch, cellulose or from glycerol and ethanol. They should preferably be coryneform bacteria of the genus Corynebacterium. The most preferred species is Corynebacterium glutamicum, which is known among experts for its ability to produce L-amino acids. Suitable bacteria include the wild-type strains:

Corynebacterium glutamicum ATCC13032;

Corynebacterium acetoglutamicum ATCC15806;

Corynebacterium acetoacidophilum ATCC13870;

Corynebacterium melassecola ATCC17965;

Corynebacterium thermoaminogenes FERM BP-1539;

Brevibacterium flavum ATCC14067;

Brevibacterium lactofermentum ATCC13869; and

Brevibacterium divaricatum ATCC 14020.

L-lysine-producing mutants or strains that may be used include:

Corynebacterium glutamicum FERM-P 1709;

Brevibacterium flavum FERM-P 1708;

Brevibacterium lactofermentum FERM-P 1712;

Corynebacterium glutamicum FERM-P 6463;

Corynebacterium glutamicum FERM-P 6464; and

Corynebacterium glutamicum DSM 5715.

An example of an L-methionine-producing strain that may be used is Corynebacterium glutamicum ATCC21608.

The nucleotide sequence of the gene which codes for the malate enzyme of Corynebacterium glutamicum can be found in the patent application FR-A-2796080. This sequence has been deposited in the databank of the National Center for Biotechnology Information (NCBI) of the National Library of Medicine (Bethesda, Md., USA) under Accession Number AF234535. The nucleotide sequence of the gene has also been reported in patent application EP1108790 as sequence nos. 3328 and 7069, and in the patent application WO0100844, see sequence no. 577 under identification code RXN10148. These sequences are also deposited in the databank of the National Center for Biotechnology Information (NCBI) of the National Library of Medicine (Bethesda, Md., USA) under Accession Number AX123412, under Accession Number AX127153 and under Accession Number AX065451.

The patent application FR 2796080 suggests that by enhancing the malate enzyme in Corynebacterium glutamicum, an increased production of amino acids, in particular lysine, is achieved. However, it has been found that, in direct contrast to the teachings of this reference, coryneform bacteria produce L-amino acids in an improved manner after the mez gene is attenuated. The sequence of the mez gene from Corynebacterium glutamicum ATCC13032, which codes for the malate enzyme, is shown in SEQ ID NO:1 and can be used according to the invention. The amino acid sequence of the protein is shown in SEQ ID NO:2. The nucleotide sequence of the mez gene as set forth in SEQ ID NO:1 differs from the sequence described in FR 2796080 at positions 138, 186, 351, 477, 483, 603, 606, 609, 612, 624, 627, 654, 657, 666, 719, 831, 837, 846, 849, 850, 1014, 1029, 1052 and 1111. The nucleobase differences at position 719 and at position 850 of the nucleotide sequence lead to changes in the amino acid sequence of the protein. Specifically, at position 240 of the amino acid sequence, aspartate is present instead of alanine and, at position 284 of the amino acid sequence, glutamine is present instead of glutamate. Alleles of the malate enzyme which result from the degeneracy of the genetic code or due to “sense mutations” of neutral function can also be used in connection with the present invention.

To achieve an attenuation, either the expression of the mez gene and/or the catalytic properties of the gene product can be reduced or eliminated. Gene expression can be reduced by suitable culturing methods or by genetic modification (mutation) of the signal structures affecting gene expression. These signal structures include, for example, repressor sequences, activator sequences, operators, promoters, attenuators, ribosome binding sites, the start codon and terminators. One skilled in the art can find a description of relevant methodology, e.g., in the patent application WO 96/15246, in Boyd and Murphy (J. Bacteriol. 170:5949 (1988)), in Voskuil and Chambliss (Nucl. Acids Res. 26:3548 (1998)), in Jensen and Hammer (Biotech. Bioeng. 58:191 (1998)), in Pátek et al. (Microbiol. 142:1297 (1999)) and in textbooks of genetics and molecular biology, such as, e.g., the textbook by Knippers (“Molekulare Genetik,” 6th edition, Georg Thieme Verlag, Stuttgart, Germany, 1995) or that by Winnacker (“Gene und Klone,” VCH Verlagsgesellschaft, Weinheim, Germany, 1990).

Another method for reducing the expression of specific genes is by the antisense technique, in which short oligodeoxynucleotides or vectors for the synthesis of antisense RNA are brought into the target cells. There, the antisense RNA can bind to complementary sections of specific mRNAs and either reduce their stability or block the rate at which they are translated. An example in this context may be found in Srivastava, et al., (Appl. Environ. Microbiol. 66(10):4366-4371 (2000)).

Mutations which lead to a change or reduction in the catalytic properties of enzyme proteins have been described in the prior art. Examples include disclosures by Qiu, et al., (J. Biol. Chem. 272:8611-8617 (1997)), Sugimoto et al. (Biosci. Biotech. Biochem. 61:1760-1762 (1997)) and Möckel (“Die Threonindehydratase aus Corynebacterium glutamicum: Aufhebung der allosterischen Regulation und Struktur des Enzyms”, Reports from the Jülich Research Center, Jül-2906, ISSN09442952, Jülich, Germany, 1994). Summaries may also be found in textbooks of genetics and molecular biology, such as, e.g., that by Hagemann (“Allgemeine Genetik”, Gustav Fischer Verlag, Stuttgart, 1986). Possible mutations are transitions, transversions, insertions and deletions of at least one base pair. Depending on the effect of the amino acid exchange on the enzyme activity, “missense mutations” or “nonsense mutations” are referred to. As a consequence of nonsense mutations, sense codons are converted into stop codons and translation terminates prematurely. Insertions or deletions of at least one base pair in a gene can lead to “frame shift mutations,” as a consequence of which incorrect amino acids are incorporated or translation is interrupted prematurely. Deletions of one or more codons typically lead to a complete loss of enzyme activity. Instructions on the generation of such mutations are well known in the prior art and can be found in textbooks of genetics and molecular biology, such as, e.g., the textbook by Knippers (“Molekulare Genetik”, 6th edition, Georg Thieme Verlag, Stuttgart, Germany, 1995), that by Winnacker (“Gene und Klone,” VCH Verlagsgesellschaft, Weinheim, Germany, 1990) or that by Hagemann (“Allgemeine Genetik”, Gustav Fischer Verlag, Stuttgart, 1986).

Common methods of mutating genes of C. glutamicum include the methods of “gene disruption” and “gene replacement” described by Schwarzer, et al. (Bio/Technology 9:84-87 (1991)). In the method of gene disruption, a central part of the coding region of the gene of interest is cloned in a plasmid vector which can replicate in a host (typically E. coli), but not in C. glutamicum. Possible vectors are, for example: pSUP301 (Simon et al., Bio/Technology 1:784-791 (1983)); pK18mob or pK19mob (Schäfer et al., Gene 145:69-73 (1994)); pK18mobsacB or pK19mobsacB (Jäger, et al., J. Bacteriol. 174:5462-65 (1992)); pGEM-T (Promega Corporation, Madison, Wis., USA); pCR2.1-TOPO (Shuman, J. Biol. Chem. 269:32678-84 (1994), U.S. Pat. No. 5,487,993); pCR®Blunt (Invitrogen, Groningen, Holland, Bernard et al., J. Mol. Biol. 234:534-541 (1993)); or pEM1 (Schrumpf, et al, J. Bacteriol. 173:4510-4516 (1991)). The plasmid vector which contains the central part of the coding region of the gene is then transferred into the desired strain of C. glutamicum by conjugation or transformation. The method of conjugation is described, for example, by Schäfer et al. (Appl. Environ. Microbiol. 60:756-759 (1994)). Methods for transformation are described, for example, by Thierbach et al. (Appl. Microbiol. Biotechnol. 29:356-362 (1988)), Dunican, et al. (Bio/Technology 7:1067-1070 (1989)) and Tauch, et al. (FEMS Microbiol. Lett. 123:343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Appl. Microbiol. Biotechnol. 42:575-580 (1994)) to eliminate the recA gene of C. glutamicum.

In the method of “gene replacement,” a mutation, such as, e.g., a deletion, insertion or base exchange, is established in vitro in the gene of interest. The allele prepared is cloned in a vector which is not replicative for C. glutamicum and this is then transferred into the desired C. glutamicum host by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch et al. (Microbiol. 144:915-927 (1998)) to eliminate the pyc gene of C. glutamicum by a deletion. A deletion, insertion or a base exchange can be incorporated into the mez gene using these procedures.

In addition to the attenuation of the mez gene, it may be advantageous for the production of L-amino acids to enhance, in particular to overexpress, one or more enzymes of the biosynthesis pathway of glycolysis, of anaplerosis, of the citric acid cycle, of the pentose phosphate cycle, of amino acid export and, optionally, regulatory proteins. The term “enhancement” or “enhance” in this context describes an increase in the intracellular activity or concentration of one or more enzymes or proteins in a microorganism which are coded for by the corresponding DNA. Enhancement may be accomplished, for example, by increasing the number of copies of the gene or genes, by using a potent promoter, by using a gene or allele which codes for a corresponding enzyme or protein with a high activity, or, optionally, by combining these measures. Using such methods the activity or concentration of the corresponding protein may be increased by at least 10%, 25%, 50%, 75%, 100%, 150%, 200%, 300%, 400% or 500%, up to a maximum of 1000% or 2000%, relative to that of the wild-type protein or to the activity or concentration of the protein in the starting, microorganism.

The use of endogenous genes is, in general, preferred. The terms “endogenous genes” or “endogenous nucleotide sequences” as used herein are understood to refer to the genes or nucleotide sequences present in the population of a species. Thus, for the preparation of L-amino acids, in addition to attenuation of the mez gene, one or more of the following genes can be enhanced, in particular over-expressed:

the lysC gene which codes for a feed-back resistant aspartate kinase (Accession No. P26512, EP-B-0387527; EP-A-0699759; WO 00/63388);

the dapA gene which codes for dihydrodipicolinate synthase (EP-B 0 197 335);

the gap gene which codes for glyceraldehyde 3-phosphate dehydrogenase (Eikmanns, J. Bacteriol. 174:6076-6086 (1992));

the pyc gene which codes for pyruvate carboxylase (EP-A-1083225);

the mqo gene which codes for malate:quinone oxidoreductase (Molenaar et al., Eur. J. Biochem. 254:395-403 (1998); EP-A-1038969);

the zwf gene which codes for glucose 6-phosphate dehydrogenase (JP-A-09224661, WO 01/70995);

the lysE gene which codes for the lysine export protein (DE-A-195 48 222);

the zwa1 gene which codes for the Zwa1 protein (EP-A-1111062);

the tpi gene which codes for triose phosphate isomerase (Eikmanns, J. Bacteriol. 174:6076-6086 (1992)); and

the pgk gene which codes for 3-phosphoglycerate kinase (Eikmanns, J. Bacteriol. 174:6076-6086 (1992)).

It may furthermore be advantageous for the production of L-amino acids, in addition to the attenuation of the mez gene, to attenuate one or more of the following genes:

the pck gene which codes for phosphoenol pyruvate carboxykinase (EP-A-1094111);

the pgi gene which codes for glucose 6-phosphate isomerase (EP-A-1087015, WO 01/07626);

the poxB gene which codes for pyruvate oxidase (EP-A-1096013);

the zwa2 gene which codes for the Zwa2 protein (EP-A-1106693); and

the ccpA1 gene which codes for a catabolite control protein A (WO 02/18419).

Finally, it may be advantageous for the production of amino acids to eliminate undesirable side reactions (see Nakayama, “Breeding of Amino Acid Producing Microorganisms,” in: Overproduction of Microbial Products, Krumphanzl, Sikyta, Vanek eds., Academic Press, London, UK, 1982).

The invention also encompasses the microorganisms prepared according to the methods described herein. These can be cultured continuously or discontinuously in a batch process (batch culture), in a fed batch (feed process) or in a repeated fed batch process (repetitive feed process) for the purpose of producing L-amino acids. A summary of culture methods is provided in the textbook by Chmiel (Bioprozesstechnik 1. Einführung in die Bioverfahrenstechnik, Gustav Fischer Verlag, Stuttgart, 1991)) and in the textbook by Storhas (Bioreaktoren und periphere Einrichtungen, Vieweg Verlag, Braunschweig/Wiesbaden, 1994).

The culture medium to be used must meet the requirements of the particular strains being fermented. Descriptions of culture media for various microorganisms are contained in the handbook “Manual of Methods for General Bacteriology” of the American Society for Bacteriology (Washington D.C., USA, 1981). Sugars and carbohydrates (e.g., glucose, sucrose, lactose, fructose, maltose, molasses, starch and cellulose), oils and fats (e.g., soya oil, sunflower oil, groundnut oil and coconut fat), fatty acids (e.g., palmitic acid, stearic acid and linoleic acid), alcohols (e.g., glycerol and ethanol), and organic acids (e.g., acetic acid) can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate, dipotassium hydrogen phosphate or the corresponding sodium-containing salts can be used as the source of phosphorus. The culture medium must furthermore comprise salts of metals, such as magnesium sulfate or iron sulfate, which are necessary for growth.

Finally, essential growth substances, such as amino acids and vitamins, can be employed in addition to the above-mentioned substances. Suitable precursors can be added to the culture medium in the form of a single batch, or can be fed in during culture.

Basic compounds, such as sodium hydroxide, potassium hydroxide, ammonia or aqueous ammonia, or acid compounds, such as phosphoric acid or sulfuric acid, can be used to control the pH of the culture. Antifoams, such as, e.g., fatty acid polyglycol esters, can be employed to control the development of foam. Substances such as, e.g., antibiotics, can be added to the medium to maintain the stability of plasmids and oxygen or oxygen-containing gas mixtures, such as, e.g., air, can be introduced into the culture to maintain aerobic conditions. The temperature of the culture should typically be 20° C. to 45° C., and preferably 25° C. to 40° C. Culturing is continued until a maximum of the desired product has formed. This target is usually reached within 10 hours to 160 hours.

Methods for assaying L-amino acids are well known in the art. Analysis can, for example, be carried out as described by Spackman et al. (Analyt. Chem. 30:1190 (1958)) by anion exchange chromatography with subsequent ninhydrin derivation, or by reversed phase HPLC, for example as described by Lindroth et al. (Analyt. Chem. 51:1167-1174 (1979)).

The invention may be further understood by reference to the following non-limiting examples.

### EXAMPLES

### Example 1

### Deletion of the mez Gene

Chromosomal DNA is isolated from the strain ATCC13032 by the method of Tauch, et al. (Plasmid 33:168-179 (1995)). On the basis of the sequence of the mez gene known for C. glutamicum from the patent application EP1108790 (sequence no. 3328 and sequence no. 7069); from patent application WO 0100844 (sequence no. 577 under Identification Code RXN10148); and from the databank of the National Center for Biotechnology Information (NCBI) of the National Library of Medicine (Bethesda, Md., USA, under Accession Number AX123412, Accession Number AX127153 and Accession Number AX065451), the following oligonucleotides are chosen for generation of the mez deletion allele by means of the polymerase chain reaction (PCR) using the “Splicing by Overlap Extension” method (Gene SOEing Method) (Horton, Mol. Biotech. 3:93-98 (1995)):

| | |
|---|---|
| Primer mez_1: | |
| 5′-ATG ACC ATC GAC CTG CAG CG-3′ | (SEQ ID NO:3) |
| Primer mez_2: | |
| 5′-AAGAAGGCGCGATGGCTGCG-3′ | (SEQ ID NO:4) |
| Primer mez_3: | |
| 5′-CGC AGC CAT CGC GCC TTC TTA ATG AGG CTT TCA CCG GCG C-3′ | (SEQ ID NO:5) |
| Primer mez_4: | |
| 5′-AAG CGT TTT GCG CTT CGG CG-3′ | (SEQ ID NO:6) |

The primers shown are synthesized by MWG Biotech (Ebersberg, Germany) and the PCR reaction is carried out using Pfu polymerase (Stratagene, product no. 600135, La Jolla, USA) and a PTC 100 Thermocycler (MJ Research Inc., Waltham, USA). The primer mez_{—}3 is composed of two regions of the nucleotide sequence which bind to nucleotides 384 to 403 and 766 to 785 within the coding sequence of mez.

Using the polymerase chain reaction, the primers mez_{—}1 and mez_{—}2 allow amplification of a DNA fragment 403 bp in size and the primers mez_{—}3 and mez_{—}4 allow amplification of a DNA fragment 432 bp in size. The amplification products are tested electrophoretically in a 0.8% agarose gel, isolated from the agarose gel with the High Pure PCR Product Purification Kit (product no. 1732676, Roche Diagnostics GmbH, Mannheim, Germany) and employed together as templates for a further PCR reaction with the primers mez_{—}1 and mez_{—}4. The mez deletion derivative 815 bp in size is generated in this manner (SEQ ID NO:7). The product amplified in this way is tested electrophoretically in a 0.8% agarose gel.

### Example 2

### Cloning of the mez Deletion Derivative in the Vector pCRBluntII

The amplified DNA fragment of 815 bp length which carries the mez deletion derivative is ligated with the Zero Blunt™ Kit of Invitrogen Corporation (Carlsbad, Calif., USA; Catalogue Number K2700-20) in the vector pCR®Blunt II (Bernard, et al., J. Mol. Biol. 234:534-541 (1993)). The E. coli strain Top10 (Grant, et al., Proc. Nat'l Acad. Sci. USA 87:4645-4649 (1990)) is then transformed with the ligation batch in accordance with the instructions of the manufacturer of the kit (Invitrogen Corporation, Carlsbad, Calif., USA). Selection for plasmid-carrying cells is made by plating out the transformation batch on LB agar (Sambrook et al., Molecular Cloning: A Laboratory Manual., 2^{nd} ed., Cold Spring Harbor Laboratory Press, Cold Spring Harbor, N.Y., 1989), supplemented with 25 mg/l kanamycin. Plasmid DNA is isolated from a transformant with the aid of the QIAprep Spin Miniprep Kit from Qiagen (Hilden, Germany) and checked by treatment with the restriction enzymes EcoRV and EcoRI followed by agarose gel electrophoresis (0.8%). The plasmid is called pCRBlunt_delmez.

### Example 3

### Construction of the Exchange Vector pK18mobsacBdeltamez

The mez deletion derivative is isolated from the plasmid pCRBlunt_delmez described in Example 2 by complete cleavage with the enzyme EcoRI. After separation in an agarose gel (0.8%) with the High Pure PCR Product Purification Kit (product no. 1732676, Roche Diagnostics GmbH, Mannheim, Germany), fragment approximately 0.83 kb in size carrying the mez deletion derivative is isolated from the agarose gel.

The mez deletion derivative obtained in this way is employed for ligation with the mobilizable cloning vector pK18mobsacB (Schäfer, et al., Gene 14:69-73 (1994)). This is cleaved completely beforehand with the restriction endonuclease EcoRI and subsequently dephosphorylated with shrimp alkaline phosphatase (Roche Diagnostics GmbH, Mannheim, Germany, Product Description SAP, product no. 1758250). The vector prepared in this way is mixed with the mez deletion allele and the mixture is treated with T4 DNA ligase (Amersham-Pharmacia, Freiburg, Germany).

The E. coli strain DH5αmcr (Grant, Proc. Nat'l Acad. Sci. USA 87:4645-4649 (1990)) is then electroporated with the ligation batch (Hanahan, In. DNA Cloning, A Practical Approach, vol. 1, ILR-Press, Cold Spring Harbor, N.Y., 1989). Selection of plasmid-carrying cells is made by plating out the transformation batch on LB agar (Sambrook et al., Molecular Cloning: A Laboratory Manual, 2^{nd} ed., Cold Spring Harbor, N.Y., 1989), which has been supplemented with 25 mg/l kanamycin.

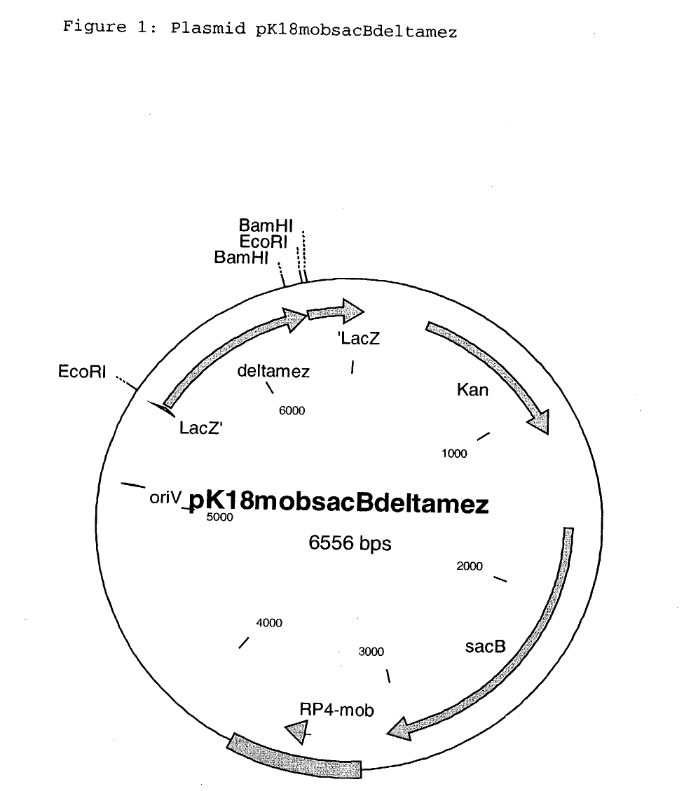

Plasmid DNA is isolated from a transformant with the aid of the QIAprep Spin Miniprep Kit from Qiagen and the cloned mez deletion allele is verified by means of restriction cleavage with the restriction endonucleases EcoRI and BamHI. The plasmid is called pK18mobsacBdeltamez and is shown in FIG. 1. The strain is called E. coli DH5αmcr/pK18mobsacBdeltamez.

### Example 4

### Deletion Mutagenesis of the mez Gene in the C. glutamicum Strain DM1637

The Corynebacterium glutamicum strain DM1637 is prepared by multiple, non-directed mutagenesis, selection and mutant selection from Corynebacterium glutamicum ATCC21527. The strain is resistant to the lysine analogue S-(2-aminoethyl)-L-cysteine and auxotrophic for the amino acids L-methionine and L-threonine.

The vector pK18mobsacBdeltamez described in Example 3 is transferred by means of conjugation using the protocol of Schäfer et al. (J. Microbiol. 172:1663-1666) (1990)) into the Corynebacterium glutamicum strain DM1637. The vector cannot replicate independently in this strain and is retained in the cells only if it has integrated into the chromosome as the consequence of a recombination event. Selection of clones with integrated pK18mobsacBdeltamez is carried out by plating the conjugation batch on LB agar (Sambrook et al., Molecular Cloning: A Laboratory Manual, 2^{nd} ed., Cold Spring Habor, N.Y., 1989), which has been supplemented with 15 mg/l kanamycin and 50 mg/ml nalidixic acid. Clones which grow are plated on LB agar plates with 25 mg/l kanamycin and incubated for 16 hours at 33° C. For selection of mutants in which excision of the plasmid has taken place as a consequence of a second recombination event, the clones are cultured unselectively for 20 hours in LB liquid medium and then plated out on LB agar with 10% sucrose and incubated for 24 hours.

The plasmid pK18mobsacBdeltamez, like the starting plasmid pK18mobsacB, contains, in addition to the kanamycin resistance gene, a copy of the sacB gene which codes for levan sucrase from Bacillus subtilis. Expression can be induced by sucrose. This leads to the formation of levan sucrase which catalyses the synthesis of the product levan, which, in turn, is toxic to C. glutamicum. Only those clones in which the integrated pK18mobsacBdeltamez has been excised therefore grow on LB agar with sucrose. In the excision, either the complete chromosomal copy of the mez gene or an incomplete copy with the internal deletion is removed.

Approximately 40 to 50 colonies are tested for the phenotype “growth in the presence of sucrose” and “non-growth in the presence of kanamycin.” To demonstrate that the deleted mez allele has remained in the chromosome, approximately 20 colonies which show the phenotype “growth in the presence of sucrose” and “non-growth in the presence of kanamycin” are investigated with the aid of the polymerase chain reaction by the standard PCR method of Innis, et al. (PCR protocols, A Guide to Methods and Applications, Academic Press, 1990). A DNA fragment which carries the mez gene and surrounding regions is amplified from the chromosomal DNA of the colonies using the following primers:

| | |
|---|---|
| mez-A1: | |
| 5′agt agc agc cca aat tca gc 3′ | (SEQ ID NO:8) |
| mez-E1: | |
| 5′ggg cct caa gtt tgc tct ta 3′ | (SEQ ID NO:9) |

The primers allow amplification of a DNA fragment approximately 1.5 kb in size in control clones with the complete mez allele. In clones with a deleted mez allele, DNA fragments with a size of approximately 1.14 kb are amplified. The amplified DNA fragments are identified by means of electrophoresis in a 0.8% agarose gel. It could thus be demonstrated that the strain DM1637 carries a deleted mez allele in its chromosome. The strain was called C. glutamicum DM1637deltamez.

### Example 5

### Preparation of Lysine

The C. glutamicum strain DM1637deltamez obtained in Example 4 is cultured in a nutrient medium suitable for the production of lysine and the lysine content in the culture supernatant is determined. To accomplish this, the strain is first incubated on an agar plate for 24 hours at 33° C. Starting from this agar plate culture, a preculture is seeded (10 ml MM medium in a 100 ml conical flask) and incubated for 24 hours at 33° C. at 240 rpm on a shaking machine. A main culture is seeded from this preculture such that the initial OD (660 nm) of the main culture is 0.1. MM medium, as shown below, is also used for the main culture.

Medium MM

| | |
|---|---|
| CSL | 5 g/l |
| MOPS | 20 g/l |
| Glucose (autoclaved separately) | 50 g/l |
| Salts: | |
| (NH₄)₂SO₄ | 25 g/l |
| KH₂PO₄ | 0.1 g/l |
| MgSO₄ * 7 H₂O | 1,0 g/l |
| CaCl₂ * 2 H₂O | 10 mg/l |
| FeSO₄ * 7 H₂O | 10 mg/l |
| MnSO₄ * H₂O | 5.0 mg/l |
| Biotin (sterile-filtered) | 0.3 mg/l |
| Thiamine * HCl (sterile-filtered) | 0.2 mg/l |
| L-Homoserine (sterile-filtered) | 0.4 g/l |
| CaCO₃ | 25 g/l |

The CSL (corn steep liquor), MOPS (morpholinopropanesulfonic acid) and the salt solution are brought to pH 7 with aqueous ammonia and autoclaved. The sterile substrate and vitamin solutions, as well as the CaCO₃ autoclaved in the dry state, are then added. Culturing is carried out in a 10 ml volume in a 100 ml conical flask with baffles at 33° C. and 80% atmospheric humidity. After 48 hours, the OD is determined at a measurement wavelength of 660 nm with a Biomek 1000 (Beckmann Instruments GmbH, Munich). The amount of lysine formed is determined with an amino acid analyzer from Eppendorf-BioTronik (Hamburg, Germany) by ion exchange chromatography and post-column derivation with ninhydrin detection. The result of the experiment is shown in Table 1.

TABLE 1

| | GD | Lysine HCl |
|---|---|---|
| Strain | (660 nm) | g/l |
| DM1637 | 10.9 | 11.3 |
| DM1637deltamez | 10.8 | 11.8 |

All references cited herein are fully incorporated by reference. Having now fully described the invention, it will be understood by those of skill in the art that the invention may be performed within a wide and equivalent range of conditions, parameters and the like, without affecting the spirit or scope of the invention or any embodiment thereof.

What is claimed is:

1. A process for the preparation of a desired L-amino acid comprising:
a) fermenting modified coryneform bacteria to produce a fermentation broth, wherein said modified coryneform bacteria produce said desired L-amino acid, and wherein the nucleotide sequence which codes for the malate enzyme (mez) in said modified coryneform bacteria is attenuated relative to unmodified or wild-type coryneform bacteria;
b) concentrating said desired L-amino acid in the fermentation broth or in the cells of said coryneform bacteria of step a); and
c) obtaining an isolated composition by purifying the desired L-amino acid concentrated in step b).

2. The process of claim 1, wherein said desired L-amino acid is L-lysine.

3. The process of claim 1, wherein said desired L-amino acid is L-methionine.

4. The process of claim 1, wherein the isolated composition produced in step c) of said process comprises, in addition to said desired amino acid, biomass from the fermentation of said modified coryneform bacteria or constituents from said fermentation broth.

5. The process of claim 1, wherein the activity of at least one gene product in the biosynthetic pathway of said desired L-amino acid is enhanced relative to the activity of the corresponding gene product in the wild type coryneform bacteria.

6. The process of claim 5, wherein the expression of the gene encoding said gene product is increased in said modified coryneform bacteria relative to expression of the corresponding gene in the wild type coryneform bacteria.

7. The process of claim 1, wherein at least one gene product in a metabolic pathway that reduces the formation of said desired amino acid is decreased in activity in said modified coryneform bacteria relative to the corresponding gene product in the wild type coryneform bacteria.

8. The process of claim 7, wherein the expression of the gene encoding said gene product is reduced in said modified coryneform bacteria relative to the corresponding gene in the wild type coryneform bacteria.

9. The process of claim 1, wherein the expression of the polynucleotide which codes for the malate enzyme is reduced.

10. The process of claim 1, wherein the catalytic properties of the polypeptide for which the polynucleotide mez codes are reduced.

11. The process of claim 1, wherein said desired L-amino acid is either L-lysine or L-methionine and, relative to wild type coryneform bacteria, said modified coryneform bacteria exhibit increased enzymatic activity or concentration of a protein selected from the group consisting of: feed-back resistant aspartate kinase, coded for by the lysC gene; dihydrodipicolinate synthase coded for by the dapA gene; glyceraldehyde 3-phosphate dehydrogenase, coded for by the gap gene; pyruvate carboxylase, coded for by the pyc gene; malate:quinone oxidoreductase, coded for by the mqo gene; glucose 6-phosphate dehydrogenase, coded for by the zwf gene; the lysine export protein, coded for by the lysE; the Zwa1 protein, coded for by the zwa1 gene; triose phosphate isomerase, coded for by the tpi gene; and 3-phosphoglycerate kinase, coded for by the pgk gene.

12. The process of claim 1 wherein said desired L-amino acid is either L-lysine or L-methionine and, relative to wild type coryneform bacteria, said modified coryneform bacteria overexpress at least one gene selected from the group consisting of: the lysC gene which codes for a feed-back resistant aspartate kinase; the dapA gene which codes for dihydrodipicolinate synthase; the gap gene which codes for glyceraldehyde 3-phosphate dehydrogenase; the pyc gene which codes for pyruvate carboxylase; the mqo gene which codes for malate:quinone oxidoreductase; the zwf gene which codes for glucose 6-phosphate dehydrogenase; the lysE gene which codes for the lysine export protein; the zwa1 gene which codes for the Zwa1 protein; the tpi gene which codes for triose phosphate isomerase; and the pgk gene which codes for 3-phosphoglycerate kinase.

13. The process of claim 1, wherein said desired L-amino acid is either L-lysine or L-methionine and, relative to wild type coryneform bacteria, said modified coryneform bacteria exhibits decreased enzymatic activity or concentration of a protein selected from the group consisting of: phosphoenolpyruvate carboxykinase, coded for by the pck gene; glucose 6-phosphate isomerase, coded for by the pgi gene; pyruvate oxidase, coded for by the poxB gene; the Zwa2 protein coded for by the zwa2 gene; and catabolite control protein A, coded for by the ccpA1 gene.

14. The process of claim 1, wherein said desired L-amino acid is either L-lysine or L-methionine and, relative to wild type coryneform bacteria, at least one gene in said modified coryneform bacteria is attenuated, said attenuated gene being selected from the group consisting of: the pgi gene which codes for glucose 6-phosphate isomerase; the poxB gene which codes for pyruvate oxidase; the zwa2 gene which codes for the Zwa2 protein; and the ccpA1 gene which codes for a catabolite control protein A.

15. The process according of any one of claims 1-14, wherein said modified coryneform bacteria and said wild type coryneform bacteria are of the species Corynebacterium glutamicum.

16. The process of claim 15, wherein said coryneform bacteria of the species Corynebacterium glutamicum comprise the plasmid pK18mobsacBdeltamez.

17. A modified coryneform bacterium comprising a polynucleotide coding for the malate enzyme (mez) that is attenuated relative to unmodified or wild-type coryneform bacteria.

18. The modified coryneform bacterium of claim 17, wherein the expression of the polynucleotide which codes for the malate enzyme is reduced.

19. The modified coryneform bacterium of claim 17, wherein the catalytic properties of the polypeptide for which the polynucleotide mez codes are reduced.

20. The modified coryneform bacterium of claim 17, wherein, relative to wild type coryneform bacteria, said modified coryneform bacterium exhibits increased enzymatic activity or concentration of a protein selected from the group consisting of: feed-back resistant aspartate kinase, coded for by the lysC gene; dihydrodipicolinate synthase coded, for by the dapA gene; glyceraldehyde 3-phosphate dehydrogenase, coded for by the gap gene; pyruvate carboxylase, coded for by the pyc gene; malate:quinone oxidoreductase, coded for by the mqo gene; glucose 6-phosphate dehydrogenase, coded for by the zwf gene; the lysine export protein, coded for by the lyse; the Zwa1 protein, coded for by the zwa1 gene; triose phosphate isomerase, coded for by the tpi gene; and 3-phosphoglycerate kinase, coded for by the pgk gene.

21. The modified coryneform bacterium of claim 17, wherein relative to wild type coryneform bacteria, said modified coryneform bacterium overexpresses at least one gene selected from the group consisting of: the lysC gene which codes for a feed-back resistant aspartate kinase; the dapA gene which codes for dihydrodipicolinate synthase; the gap gene which codes for glyceraldehyde 3-phosphate dehydrogenase; the pyc gene which codes for pyruvate carboxylase; the mqo gene which codes for malate:quinone oxidoreductase; the zwf gene which codes for glucose 6-phosphate dehydrogenase; the lysE gene which codes for the lysine export protein; the zwa1 gene which codes for the Zwa1 protein; the tpi gene which codes for triose phosphate isomerase; and the pgk gene which codes for 3-phosphoglycerate kinase.

22. The modified coryneform bacterium of claim 17, wherein relative to wild type coryneform bacteria, said modified coryneform bacterium exhibits decreased enzymatic activity or concentration of a protein selected from the group consisting of: phosphoenolpyruvate carboxykinase, coded for by the pck gene; glucose 6-phosphate isomerase, coded for by the pgi gene; pyruvate oxidase, coded for by the poxB gene; the Zwa2 protein coded for by the zwa2 gene; and catabolite control protein A, coded for by the ccpA1 gene.

23. The modified coryneform bacterium of claim 17, wherein, relative to wild type coryneform bacteria, at least one gene in said modified coryneform bacterium is attenuated, said attenuated gene being selected from the group consisting of: the pgi gene which codes for glucose 6-phosphate isomerase; the poxB gene which codes for pyruvate oxidase; the zwa2 gene which codes for the Zwa2 protein; and the ccpA1 gene which codes for a catabolite control protein A.

24. The modified coryneform bacterium of any one of claims 17-23, wherein said modified coryneform bacterium and said wild type coryneform bacteria are of the species Corynebacterium glutamicum.

25. The modified coryneform bacterium of claim 17, wherein said modified coryneform bacterium of the species Corynebacterium glutamicum comprise the plasmid pK18mobsacBdeltamez.


SEQUENCE LISTING (9 sequences)

SEQ ID NO: 1 - DNA, 1179 bp, Corynebacterium glutamicum
  CDS (1)..(1176): mez gene
atgaccatcgacctgcagcgttccacccaaaacctcacccatgaggaaatcttcgaggcacacgagggcggaaagctctccattagttccactcgtccgctccgcgacatgcgcgatctttcccttgcttacacccctggtgttgctcaggtttgtgaagcaatcaaggaagatccagaggttgcgcgcacccacacgggcattggaaacaccgtcgcggttatttccgacggcaccgctgttcttggccttggcgatatcggacctcaggcctcccttcccgtcatggagggcaaggctcagctgtttagctctttcgctggcctgaaggctatccctatcgttttggacgttcacgatgttgacgctttggttgagaccatcgcagccatcgcgccttctttcggtgctatcaacttggaggacatctccgctcctcgttgcttcgaggtggagcgccgcctcatcgagcgtctcgatattccagttatgcacgatgaccagcacggcaccgctgtggttatcctcgctgcgctgcgcaactccctgaagctgctggatcgcaagatcgaagacctcaagattgttatttccggcgcaggcgcagcgggcgttgcagctgtagatatgctgaccaacgctggagcaaccgacatcgtggttcttgattcccgaggcatcatccacgacagccgtgaggatctttccccagttaaggctgctcttgcagagaagaccaaccctcgtggcatcagcggtggcatcaatgaggctttcaccggcgcggacctgttcattggcgtgtccggcggcaacatcggcgaggacgctctcaaactcatggccccggagccaatcctgttcaccctggcgaacccaaccccagagatcgatcctgagctgtctcagaagtacggcgccatcgtcgcgaccggccgctctgacctgcctaaccagatcaacaacgtgctcgcgttcccaggaattttcgccggcgctctcgcagccaaggctaagaagatcacccccgagatgaagctcgccgctgcagaggcaatcgccgacatcgcagctgaggacctcgaggtcggccgcatcgtgcctaccgccctggatccccgcgtcgccccagcagtcaaggcagctgtccaggccgtcgccgaagcgcaaaacgcttaa

SEQ ID NO: 2 - protein, 392 aa, Corynebacterium glutamicum
MTIDLQRSTQNLTHEEIFEAHEGGKLSISSTRPLRDMRDLSLAYTPGVAQVCEAIKEDPEVARTHTGIGNTVAVISDGTAVLGLGDIGPQASLPVMEGKAQLFSSFAGLKAIPIVLDVHDVDALVETIAAIAPSFGAINLEDISAPRCFEVERRLIERLDIPVMHDDQHGTAVVILAALRNSLKLLDRKIEDLKIVISGAGAAGVAAVDMLTNAGATDIVVLDSRGIIHDSREDLSPVKAALAEKTNPRGISGGINEAFTGADLFIGVSGGNIGEDALKLMAPEPILFTLANPTPEIDPELSQKYGAIVATGRSDLPNQINNVLAFPGIFAGALAAKAKKITPEMKLAAAEAIADIAAEDLEVGRIVPTALDPRVAPAVKAAVQAVAEAQNA

SEQ ID NO: 3 - DNA, 20 bp, Corynebacterium glutamicum
  misc_feature (1)..(20): Primer mez_1
atgaccatcgacctgcagcg

SEQ ID NO: 4 - DNA, 20 bp, Corynebacterium glutamicum
  misc_feature (1)..(20): Primer mez_2
aagaaggcgcgatggctgcg

SEQ ID NO: 5 - DNA, 40 bp, Artificial sequence
  misc_feature (1)..(40): Primer mez_3
cgcagccatcgcgccttcttaatgaggctttcaccggcgc

SEQ ID NO: 6 - DNA, 20 bp, Corynebacterium glutamicum
  misc_feature (1)..(20): Primer mez_4
aagcgttttgcgcttcggcg

SEQ ID NO: 7 - DNA, 815 bp, Artificial sequence
  misc_feature (1)..(815): PCR product mez deletion derivative
atgaccatcgacctgcagcgttccacccaaaacctcacccatgaggaaatcttcgaggcacacgagggcggaaagctctccattagttccactcgtccgctccgcgacatgcgcgatctttcccttgcttacacccctggtgttgctcaggtttgtgaagcaatcaaggaagatccagaggttgcgcgcacccacacgggcattggaaacaccgtcgcggttatttccgacggcaccgctgttcttggccttggcgatatcggacctcaggcctcccttcccgtcatggagggcaaggctcagctgtttagctctttcgctggcctgaaggctatccctatcgttttggacgttcacgatgttgacgctttggttgagaccatcgcagccatcgcgccttcttaatgaggctttcaccggcgcggacctgttcattggcgtgtccggcggcaacatcggcgaggacgctctcaaactcatggccccggagccaatcctgttcaccctggcgaacccaaccccagagatcgatcctgagctgtctcagaagtacggcgccatcgtcgcgaccggccgctctgacctgcctaaccagatcaacaacgtgctcgcgttcccaggaattttcgccggcgctctcgcagccaaggctaagaagatcacccccgagatgaagctcgccgctgcagaggcaatcgccgacatcgcagctgaggacctcgaggtcggccgcatcgtgcctaccgccctggatccccgcgtcgccccagcagtcaaggcagctgtccaggccgtcgccgaagcgcaaaacgctt

SEQ ID NO: 8 - DNA, 20 bp, Corynebacterium glutamicum
  misc_feature (1)..(20): Primer mez-A1
agtagcagcccaaattcagc

SEQ ID NO: 9 - DNA, 20 bp, Corynebacterium glutamicum
  misc_feature (1)..(20): Primer mez-E1
gggcctcaagtttgctctta